SEQUENCE LISTING (14 sequences)

SEQ ID NO: 1 - RNA, 25 nt, Homo sapiens
  source 1..25: mol_type = mRNA
cgagcagggtatcgacgattacaaa

SEQ ID NO: 2 - RNA, 25 nt, Homo sapiens
  source 1..25: mol_type = other RNA
tttgtaatcgtcgataccctgctcg

SEQ ID NO: 3 - RNA, 21 nt, synthetic construct
  source 1..21: mol_type = other RNA
  modified_base 19: mod_base = OTHER; note = 2'-O-methyl-adenosine
  modified_base 8: mod_base = OTHER; note = 2'-O-methyl-adenosine
  modified_base 7: mod_base = OTHER; note = 2'-fluoro-guanosine
  modified_base 20: mod_base = OTHER; note = 2'-O-methyl-adenosine
  modified_base 2: mod_base = OTHER; note = 2'-O-methyl-adenosine
  modified_base 4: mod_base = OTHER; note = 2'-O-methyl-adenosine
  modified_base 3: mod_base = gm; note = 2'-O-methyl-guanosine
  modified_base 6: mod_base = OTHER; note = 2'-O-methyl-adenosine
  modified_base 10: mod_base = OTHER; note = 2'-fluoroadenosine
  modified_base 18: mod_base = OTHER; note = 2',2'-difluoro-2'-deoxycytidine
  modified_base 11: mod_base = OTHER; note = 2'-fluorouracil
  modified_base 9: mod_base = OTHER; note = 2'-fluoro-guanosine
  modified_base 14: mod_base = gm; note = 2'-O-methyl guanosine
  modified_base 5: mod_base = OTHER; note = 2'-O-methyl-adenosine
  modified_base 12: mod_base = OTHER; note = 2',2'-difluoro-2'-deoxycytosine
  modified_base 8: mod_base = OTHER; note = 2'-O-methyl-adenosine
  modified_base 1: mod_base = OTHER; note = 2'-O-methyl-adenosine
  modified_base 15: mod_base = um
  modified_base 13: mod_base = um
  modified_base 16: mod_base = OTHER; note = 2'-O-methyl-adenosine
  modified_base 17: mod_base = um
  modified_base 21: mod_base = um
aagaaagagattcgtatcaat

SEQ ID NO: 4 - RNA, 25 nt, synthetic construct
  source 1..25: mol_type = other RNA
  modified_base 1: mod_base = OTHER; note = 2'-fluorouracil
  modified_base 3: mod_base = um
  modified_base 4: mod_base = gm
  modified_base 5: mod_base = OTHER; note = 2'-O-methyl adenosine
  modified_base 6: mod_base = OTHER; note = 2'-fluorouracil
  modified_base 7: mod_base = OTHER; note = 2'-O-methyl adenosine
  modified_base 8: mod_base = cm
  modified_base 9: mod_base = OTHER; note = 2'-O-methyl adenosine
  modified_base 10: mod_base = gm
  modified_base 11: mod_base = OTHER; note = 2'-O-methyl adenosine
  modified_base 12: mod_base = um
  modified_base 13: mod_base = cm
  modified_base 14: mod_base = OTHER; note = 2'-fluorouracil
  modified_base 15: mod_base = cm
  modified_base 16: mod_base = OTHER; note = 2'-fluorouracil
  modified_base 17: mod_base = OTHER; note = 2'-fluorouracil
  modified_base 17: mod_base = OTHER; note = 2'-fluorouracil
  modified_base 18: mod_base = OTHER; note = 2'-fluorouracil
  modified_base 19: mod_base = OTHER; note = 2'-fluorocytosine
  modified_base 20: mod_base = OTHER; note = 2'-fluorouracil
  modified_base 21: mod_base = OTHER; note = 2'-fluorouracil
  modified_base 22: mod_base = OTHER; note = 2',2'-difluorodeoxycytidine
  modified_base 23: mod_base = OTHER; note = 2',2'-difluorodeoxycytidine
  modified_base 2: mod_base = OTHER; note = 2'-fluorouracil
  modified_base 24: mod_base = OTHER; note = thymine
  modified_base 25: mod_base = OTHER; note = thymine
attgatacagatctctttcttcctt

SEQ ID NO: 5 - RNA, 21 nt, synthetic construct
  source 1..21: mol_type = other RNA
  modified_base 7: mod_base = OTHER; note = 2'-fluoro-guanosine
  modified_base 4: mod_base = OTHER; note = 2'-O-methyl adenosine
  modified_base 5: mod_base = OTHER; note = 2'-O-methyl adenosine
  modified_base 3: mod_base = gm
  modified_base 10: mod_base = OTHER; note = 2'-fluoroadenosine
  modified_base 8: mod_base = OTHER; note = 2'-O-methyl adenosine
  modified_base 9: mod_base = OTHER; note = 2'-fluoro-guanosine
  modified_base 6: mod_base = OTHER; note = 2'-O-methyl adenosine
  modified_base 2: mod_base = OTHER; note = 2'-O-methyl adenosine
  modified_base 1: mod_base = OTHER; note = 2'-O-methyl adenosine
  modified_base 11: mod_base = OTHER; note = 2'-fluorouracil
  modified_base 12: mod_base = OTHER; note = 2',2'-difluorocytidine
  modified_base 18: mod_base = OTHER; note = 2',2'-difluorocytidine
  modified_base 16: mod_base = OTHER; note = 2'-O-methyl adenosine
  modified_base 14: mod_base = gm
  modified_base 21: mod_base = OTHER; note = 2'-fluorouracil
  modified_base 13: mod_base = OTHER; note = 2'-fluorouracil
  modified_base 15: mod_base = OTHER; note = 2'-fluorouracil
  modified_base 17: mod_base = OTHER; note = 2'-fluorouracil
  modified_base 19: mod_base = OTHER; note = 2'-O-methyl-adenosine
  modified_base 20: mod_base = OTHER; note = 2'-fluoroadenosine
aagaaagagatctgtatcaat

SEQ ID NO: 6 - RNA, 25 nt, synthetic construct
  source 1..25: mol_type = other RNA
  modified_base 1: mod_base = OTHER; note = 2'-O-methyl adenosine
  modified_base 3: mod_base = um
  modified_base 4: mod_base = gm
  modified_base 5: mod_base = OTHER; note = 2'-O-methyl adenosine
  modified_base 6: mod_base = OTHER; note = 2'-fluorouracil
  modified_base 7: mod_base = OTHER; note = 2'-O-methyl adenosine
  modified_base 8: mod_base = cm
  modified_base 9: mod_base = OTHER; note = 2'-O-methyl adenosine
  modified_base 10: mod_base = gm
  modified_base 11: mod_base = OTHER; note = 2'-O-methyl adenosine
  modified_base 12: mod_base = OTHER; note = 2'-fluorouracil
  modified_base 13: mod_base = cm
  modified_base 14: mod_base = OTHER; note = 2'-fluorouracil
  modified_base 15: mod_base = cm
  modified_base 16: mod_base = OTHER; note = 2'-fluorouracil
  modified_base 17: mod_base = um
  modified_base 18: mod_base = OTHER; note = 2'-fluorouracil
  modified_base 19: mod_base = cm
  modified_base 20: mod_base = OTHER; note = 2'-fluorouracil
  modified_base 21: mod_base = um
  modified_base 22: mod_base = OTHER; note = 2',2'-difluorodeoxycytidine
  modified_base 23: mod_base = OTHER; note = 2',2'-difluorodeoxycytidine
  modified_base 22: mod_base = OTHER; note = 2',2'-difluorodeoxycytidine
  modified_base 2: mod_base = OTHER; note = 2'-fluorouracil
  modified_base 24: mod_base = OTHER; note = thymine
  modified_base 25: mod_base = OTHER; note = thymine
attgatacagatctctttcttcctt

SEQ ID NO: 7 - RNA, 21 nt, synthetic construct
  source 1..21: mol_type = other RNA
  modified_base 18: mod_base = OTHER; note = 2'-fluorocytosine
  modified_base 10: mod_base = OTHER; note = 2'-fluoroadenosine
  modified_base 14: mod_base = gm
  modified_base 11: mod_base = OTHER; note = 2'-fluorouracil
  modified_base 13: mod_base = um
  modified_base 3: mod_base = gm
  modified_base 4: mod_base = OTHER; note = 2'-O-methyl adenosine
  modified_base 7: mod_base = OTHER; note = 2'-fluoroguanosine
  modified_base 2: mod_base = OTHER; note = 2'-O-methyl adenosine
  modified_base 19: mod_base = OTHER; note = 2'-O-methyl adenosine
  modified_base 13: mod_base = um
  modified_base 5: mod_base = OTHER; note = 2'-O-methyl adenosine
  modified_base 8: mod_base = OTHER; note = 2'-O-methyl adenosine
  modified_base 1: mod_base = OTHER; note = 2'-O-methyl adenosine
  modified_base 9: mod_base = OTHER; note = 2'-fluoroguanosine
  modified_base 20: mod_base = OTHER; note = 2'-O-methyl adenosine
  modified_base 15: mod_base = um
  modified_base 21: mod_base = um
  modified_base 12: mod_base = OTHER; note = 2'-fluorocytosine
  modified_base 17: mod_base = um; mod_base = um
  modified_base 6: mod_base = OTHER; note = 2'-O-methyl-adenosine
aagaaagagatctgtatcaat

SEQ ID NO: 8 - RNA, 23 nt, synthetic construct
  source 1..23: mol_type = other RNA
  modified_base 1: mod_base = OTHER; note = 2'-O-methyl adenosine
  modified_base 3: mod_base = um
  modified_base 4: mod_base = gm
  modified_base 5: mod_base = OTHER; note = 2'-O-methyl adenosine
  modified_base 6: mod_base = OTHER; note = 2'-fluorouracil
  modified_base 7: mod_base = OTHER; note = 2'-O-methyl adenosine
  modified_base 8: mod_base = cm
  modified_base 9: mod_base = OTHER; note = 2'-O-methyl adenosine
  modified_base 10: mod_base = gm
  modified_base 11: mod_base = OTHER; note = 2'-O-methyl adenosine
  modified_base 12: mod_base = um
  modified_base 13: mod_base = cm
  modified_base 14: mod_base = OTHER; note = 2'-fluorouracil
  modified_base 15: mod_base = cm
  modified_base 16: mod_base = OTHER; note = 2'-fluorouracil
  modified_base 16: mod_base = OTHER; note = 2'-fluorouracil
  modified_base 17: mod_base = OTHER; note = 2'-fluorouracil
  modified_base 18: mod_base = OTHER; note = 2'-fluorouracil
  modified_base 19: mod_base = cm
  modified_base 20: mod_base = OTHER; note = 2'-fluorouracil
  modified_base 21: mod_base = OTHER; note = 2'-fluorouracil
  modified_base 22: mod_base = OTHER; note = thymine
  modified_base 2: mod_base = OTHER; note = 2'-fluorouracil
  modified_base 23: mod_base = OTHER; note = thymine
attgatacagatctctttctttt

SEQ ID NO: 9 - RNA, 21 nt, synthetic construct
  source 1..21: mol_type = other RNA
  modified_base 15: mod_base = OTHER; note = 2'-fluorouracil
  modified_base 3: mod_base = gm
  modified_base 11: mod_base = OTHER; note = 2'-fluorouracil
  modified_base 4: mod_base = OTHER; note = 2'-O-methyl adenosine
  modified_base 8: mod_base = OTHER; note = 2'-O-methyl adenosine
  modified_base 2: mod_base = OTHER; note = 2'-O-methyl adenosine
  modified_base 1: mod_base = OTHER; note = 2'-O-methyl adenosine
  modified_base 14: mod_base = gm
  modified_base 13: mod_base = OTHER; note = 2'-fluorouracil
  modified_base 7: mod_base = OTHER; note = 2'-fluoro-guanosine
  modified_base 6: mod_base = OTHER; note = 2'-O-methyl adenosine
  modified_base 21: mod_base = OTHER; note = 2'fluorouracil
  modified_base 12: mod_base = OTHER; note = 2'-fluorocytosine
  modified_base 5: mod_base = OTHER; note = 2'-O-methyl adenosine
  modified_base 9: mod_base = OTHER; note = 2'-fluoro-guanosine
  modified_base 18: mod_base = OTHER; note = 2'-fluorocytosine
  modified_base 16: mod_base = OTHER; note = 2'-O-methyl adenosine
  modified_base 10: mod_base = OTHER; note = 2'-fluoroadenosine
  modified_base 20: mod_base = OTHER; note = 2'-O-methyl adenosine
  modified_base 19: mod_base = OTHER; note = 2'-O-methyl adenosine
  modified_base 17: mod_base = OTHER; note = 2'-fluorouracil
aagaaagagatctgtatcaat

SEQ ID NO: 10 - RNA, 23 nt, synthetic construct
  source 1..23: mol_type = other RNA
  modified_base 3: mod_base = um
  modified_base 1: mod_base = OTHER; note = 2'-O-methyl adenosine
  modified_base 4: mod_base = gm
  modified_base 5: mod_base = OTHER; note = 2'-O-methyl adenosine
  modified_base 6: mod_base = OTHER; note = 2'-fluorouracil
  modified_base 7: mod_base = OTHER; note = 2'-O-methyl adenosine
  modified_base 8: mod_base = cm
  modified_base 9: mod_base = OTHER; note = 2'-O-methyl adenosine
  modified_base 10: mod_base = gm
  modified_base 11: mod_base = OTHER; note = 2'-O-methyl adenosine
  modified_base 12: mod_base = OTHER; note = 2'-fluorouracil
  modified_base 13: mod_base = cm
  modified_base 14: mod_base = OTHER; note = 2'-fluorouracil
  modified_base 15: mod_base = cm
  modified_base 16: mod_base = OTHER; note = 2'-fluorouracil
  modified_base 17: mod_base = um
  modified_base 18: mod_base = OTHER; note = 2'-fluorouracil
  modified_base 19: mod_base = cm
  modified_base 20: mod_base = OTHER; note = 2'-fluorouracil
  modified_base 21: mod_base = um
  modified_base 22: mod_base = OTHER; note = thymine
  modified_base 23: mod_base = OTHER; note = thymine
  modified_base 2: mod_base = OTHER; note = 2'-fluorouracil
attgatacagatctctttctttt

SEQ ID NO: 11 - RNA, 21 nt, synthetic construct
  source 1..21: mol_type = other RNA
  modified_base 1: mod_base = OTHER; note = 2'-O-methyl adenosine
  modified_base 9: mod_base = OTHER; note = 2'-fluoro-guanosine
  modified_base 15: mod_base = OTHER; note = 2'-fluorouracil
  modified_base 21: mod_base = OTHER; note = 2'-fluorouracil
  modified_base 16: mod_base = OTHER; note = 2'-O-methyl adenosine
  modified_base 6: mod_base = OTHER; note = 2'-O-methyl adenosine
  modified_base 17: mod_base = OTHER; note = 2'-fluorouracil
  modified_base 19: mod_base = OTHER; note = 2'-O-methyl adenosine
  modified_base 5: mod_base = OTHER; note = 2'-O-methyl adenosine
  modified_base 14: mod_base = gm
  modified_base 10: mod_base = OTHER; note = 2'-fluoroadenosine
  modified_base 12: mod_base = OTHER; note = 2',2'-difluorodeoxycytidine
  modified_base 13: mod_base = OTHER; note = 2'-fluorouracil
  modified_base 7: mod_base = OTHER; note = 2'-fluoro-guanosine
  modified_base 11: mod_base = OTHER; note = 2'-fluorouracil
  modified_base 4: mod_base = OTHER; note = 2'-O-methyl adenosine
  modified_base 2: mod_base = OTHER; note = 2'-O-methyl adenosine
  modified_base 20: mod_base = OTHER; note = 2'-O-methyl adenosine
  modified_base 8: mod_base = OTHER; note = 2'-O-methyl adenosine
  modified_base 3: mod_base = OTHER; note = gm
  modified_base 18: mod_base = OTHER; note = 2',2'-difluorodeoxycytidine
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
aagaaagagatctgtatcaat

SEQ ID NO: 12 - RNA, 27 nt, synthetic construct
  source 1..27: mol_type = other RNA
  modified_base 3: mod_base = um
  modified_base 1: mod_base = OTHER; note = 2'-O-methyl adenosine
  modified_base 4: mod_base = gm
  modified_base 5: mod_base = OTHER; note = 2'-O-methyl adenosine
  modified_base 6: mod_base = OTHER; note = 2'-fluorouracil
  modified_base 7: mod_base = OTHER; note = 2'-O-methyl adenosine
  modified_base 8: mod_base = cm
  modified_base 9: mod_base = OTHER; note = 2'-O-methyl adenosine
  modified_base 10: mod_base = gm
  modified_base 11: mod_base = OTHER; note = 2'-O-methyl adenosine
  modified_base 11: mod_base = OTHER; note = 2'-O-methyl adenosine
  modified_base 12: mod_base = OTHER; note = 2'-fluorouracil
  modified_base 13: mod_base = cm
  modified_base 14: mod_base = OTHER; note = 2'-fluorouracil
  modified_base 15: mod_base = cm
  modified_base 9: mod_base = OTHER; note = 2'-O-methyl adenosine
  modified_base 16: mod_base = OTHER; note = 2'-fluorouracil
  modified_base 17: mod_base = um
  modified_base 18: mod_base = OTHER; note = 2'-fluorouracil
  modified_base 19: mod_base = cm
  modified_base 20: mod_base = OTHER; note = 2'-fluorouracil
  modified_base 21: mod_base = um
  modified_base 23: mod_base = OTHER; note = 2',2'-difluorodeoxycytidine
  modified_base 22: mod_base = OTHER; note = 2',2'-difluorodeoxycytidine
  modified_base 25: mod_base = OTHER; note = thymine
  modified_base 1: mod_base = OTHER; note = 2'-O-methyl adenosine
  modified_base 2: mod_base = OTHER; note = 2'-fluorouracil
  modified_base 24: mod_base = OTHER; note = thymine
  modified_base 1^2: mod_base = OTHER; note = phosphorothioate
  modified_base 2^3: mod_base = OTHER; note = phosphorothioate
  modified_base 19^20: mod_base = OTHER; note = phosphorothioate
  modified_base 20^21: mod_base = OTHER; note = phosphorothioate
attgatacagatctctttcttccdtdt

SEQ ID NO: 13 - RNA, 19 nt, Homo sapiens
  source 1..19: mol_type = other RNA
cgctctgtcagccttacta

SEQ ID NO: 14 - RNA, 25 nt, Homo sapiens
  source 1..25: mol_type = other RNA
gcgtggaaagcgtagacaaggagat

(19) United States
(12) Patent Application Publication
(10) Pub. No.: US 2026/0265754 A1
(43) Pub. Date: Sep. 10, 2026

(54) CHK1-TARGETING SIRNAS CHEMICALLY MODIFIED WITH GEMCITABINE

(71) Applicant: Sirnaomics, Inc., Germantown, MD (US)

(72) Inventors: Bhaskar HALAMI, Germantown, MD (US); Vera SIMONENKO, Germantown, MD (US); Wookhyun KIM, Germantown, MD (US); Daniel MUTISYA, Germantown, MD (US); David M. EVANS, Germantown, MD (US)

(21) Appl. No.: 19/098,292

(22) Filed: Apr. 2, 2025

Related U.S. Application Data
(60) Provisional application No. 63/573,458, filed on Apr. 2, 2024.

(51) Int. Cl. C12N 15/113; A61P 35/00

(52) U.S. Cl. CPC C12N 15/1137; A61P 35/00; C12Y 207/10002; C12Y 207/11001; C12N 2310/14; C12N 2310/315; C12N 2310/321; C12N 2310/322; C12N 2310/334; C12N 2320/31; C12N 2320/32

(57) ABSTRACT

Compositions comprising uniquely methyl- and fluoro-modified, Chk1-targeting siRNAs incorporating one or more gemcitabine molecules are provided. Methods of formulating the compositions also are provided, together with methods for using the composition for treating a variety of cancers and other disorders and diseases in mammals.

This application claims priority to U.S. provisional application No. 63/573,458, filed Apr. 2, 2024, the contents of which are hereby incorporated by reference in their entirety.

### SEQUENCE LISTING

The instant application contains a Sequence Listing which has been submitted electronically in XML format and is hereby incorporated by reference in its entirety. Said XML document, created on May 15, 2026, is named 46900099CMay152026 and is 134,713 bytes in size.

### FIELD

Compositions containing small molecule, modified CHK1 inhibitors with gemcitabine are provided, together with methods for their manufacture and their use against triple negative breast cancer (TNBC), pancreatic cancer and other cancers.

### BACKGROUND

In previous work by others, it was demonstrated that silencing or inhibiting CHK1 could augment the activity of gemcitabine (2′-deoxy-2′,2′-difluorocytidine monohydrochloride) in a number of tumor cell types ([2-4]). Based on these observations it was previously reported that gemcitabine amidites could be directly incorporated into the sequence of an oligonucleotide encoding an siRNA sequence against CHK1. These constructs demonstrated improved efficacy of the product in pancreatic tumor cells [1]. Specifically, gemcitabine cold be substituted in place of cytidine residues in the siRNA sense strand (SS). Two gemcitabine moieties per sense strand along with two at the 3′ end of the antisense strand (AS) gave optimal synergistic effect in reducing the cell viability of pancreatic tumor cells in vitro [1]. The constructs retained the activity of gemcitabine (inducing RRM1, and RRM2 activity) and CHK1 inhibition (inducing gamma H2AX) through silencing of CHK1. The transfection of this siRNA construct into pancreatic tumor cells (using lipofectamine) produced a synergistic effect on cell killing by the combination not seen with either agent alone and the constructs demonstrated improved potency and efficacy compared with gemcitabine or CHK1 siRNA alone.

The constructs reported previously were synthesized using native bases (chemically unmodified). It has been shown that chemically modifying the bases (using 2′-Fluoro or 2′-Methoxy modifications) in an siRNA can reduce off target effects and can also result in prolonged stability of the siRNA with longer silencing effects [5]. The effect of chemically modifying the siRNA backbone was compared with the unmodified construct and it was shown that the products demonstrated a further increase in potency in all cells tested. The constructs were shown to inhibit other tumor types including ovarian, bladder, TNBC as well as lung cancer and the product demonstrated improved efficacy and potency in all cell types studied.

Finally, a construct that showed activity in vitro was tested using lipofectamine transfection as well as after formulation using polypeptide nanoparticles and the nanoparticle constructs were tested in vivo using a pancreatic tumor xenograft model. The product demonstrated inhibitory effects in vivo with no effect on the body weights of the animals. This formulation is useful as a therapeutic to treat cancer.

Adding gemcitabine molecules into the siRNA demonstrates improved potency, not only against pancreatic cancer cell models but also against lung cancer, bladder cancer, ovarian cancer, TNBC, and colon cancer.

Different versions of CHK1 siRNA+gemcitabine into polypeptide nanoparticles were formulated with good control over size and zeta potential. When delivered IV to mice bearing a pancreatic xenograft tumor, the cocktail showed a significant reduction in tumor burden. The product demonstrated inhibitory effects in vivo, with no effect on the body weights of the animals.

The potency of this reagent was further improved by including additional siRNAs targeting either WEE1 or BCL-XL and it was found that, in vitro, the pancreatic cancer cell line (MiaPaca2) and the TNBC cell line (MDA-MB-231) are particularly sensitive to the combination of CHK1 siRNA and gemcitabine plus a second siRNA against these targets.

### SUMMARY

Disclosed herein are pharmaceutical compositions comprising chemically modified, Chk1-targeting interfering RNA constructs containing one or more gemcitabine moieties in place of cytidine, formulated with branched histidine-lysine polymers to form peptide nanoparticles (PNP). The interfering RNA constructs may be any of siRNA, miRNA, and shRNA and mRNA. The chemical modifications include a 2′-OMe modification a 2′-Fluoro modification and a phosphorothioate modification, and the histidine-lysine polymers may be a branched polymer such as HKP or HKP(+H). Also provided are pharmaceutical composition embodiments comprising siRNA constructs selected from SEQ ID Nos. 3-4, 5-6, 7-8, 9-10 and 11-12. In other embodiments at least one of two of the interfering RNA molecules is selected from SEQ ID Nos. 13 and 14. In other embodiments the composition comprises lipofectamine.

Also provided are methods of manufacture of the interfering RNA molecules and the peptide nanoparticles (PNP), as well as methods of treating a variety of cancers in a subject, including pancreatic, triple negative breast cancer, lung, colon, ovarian and urinary bladders cancer. In some embodiments a method of treatment is provided for treating a mammal, including a human subject, having cancer, in which a pharmaceutical composition is administered intravenously and comprises a double stranded siRNA construct selected from SEQ ID Nos. 3-4, 5-6, 7-8, 9-10, and 11-12; in still other method of treatment embodiments the siRNA at least one of SEQ ID No. 13 or SEQ ID No. 14 is selected. In still other method of treatment embodiments, lipofectamine is administered as a component of the composition. In other method of treatment embodiments a subject is administered the pharmaceutical composition intratumorally or subcutaneously. In one method of treatment embodiment, a subject is administered a pharmaceutical composition comprising an anti-Chk1-gem siRNA construct having the sequence of SEQ ID Nos. 5-6 and SEQ ID No. 13; in another embodiment SEQ ID Nos. 5-6 are combined with SEQ ID No. 14. In still other method of treatment embodiments the subject's expression of a targeted gene such as Chk1 is reduced between 20 and 100 percent. In other method of treatment embodiments, the subject's tumor volume is reduced or cancer cell levels are reduced by 20 to 100 percent.

### BRIEF DESCRIPTION OF THE DRAWINGS

FIG. 1 shows the effect of the modified Chk1-targeting siRNAs with and without gemcitabine to reduce viability of pancreatic cancer (MiaPaCa-2) cells after lipofectamine transfection at varying concentrations.

FIGS. 2A-2L show the effect of the modified Chk1-targeting gemcitabine siRNAs to reduce viability of cancer in cells of varying types. Specifically, FIGS. 2A-2E show the effectiveness of PGN7+NS and WEE1+NS individually as well as PGN7+WEE1 in combination to reduce cell viability in the following types of cells: FIG. 2A: Panc 10.05 (G12D); FIG. 2B: H358 (KRAS G12C); FIG. 2C: LS180 (G12D/wt); FIG. 2D: MiaPaCa-2 (KRAS G12C); and FIG. 2E: H1299 (KRAS wt). “NS” refers to a non-silencing siRNA control. FIGS. 2F-2J show the effectiveness of PGN7+NS and BCLxL+NS individually as well as PGN7+BCLxL in combination to reduce cell viability in the following types of cells: FIG. 2F: Panc 10.05 (G12D); FIG. 2G: H358 (KRAS G12C); FIG. 2H: LS180 (G12D/wt); FIG. 2I: MiaPaCa-2 (KRAS G12C); and FIG. 2J: H1299 (KRAS wt). FIGS. 2K and 2L show the effectiveness of the modified CHkl-targeting siRNAs with and without gemcitabine to reduce cell viability in MDA-MB-231 cells (FIG. 2K, metastatic breast cancer cell line), and in OVCAR-3 cells (FIG. 2L, ovarian cancer cell line).

FIGS. 3A-3C show characteristics of the PNP formed from the formulation of siRNAs and a branched histidine lysine polymer (HKP (+H)). FIG. 3A shows the size and low polydispersity index (PDI) of PNP. FIG. 3B shows dynamic light scattering using a Malvern/Wyatt instrument. FIG. 3C shows the results from a gel retardation assay when the PNP/siRNA formulation was evaluated.

FIGS. 4A-4D show the stability of chemically modified and unmodified Chk1-Gem siRNA in human serum. FIG. 4A shows the native PAGE analysis results of evaluating the stability of the chemically modified and unmodified Chk1-Gem siRNA in zero or 50 percent human serum, incubated for 1, 4 or 18 hours (versus control). FIG. 4B shows the results of gel assay for the various siRNA constructs after exposure to 90 percent human serum following 1, 4 or 18 hours of incubation. FIGS. 4C and 4D shows cell viability in MiaPaCa2 cells (FIG. 4C) and MDA-MB-231 cells (FIG. 4D) using a series of siRNA concentrations (0.5, 5, 10, 50 and 100 nM) of PGN6 with HKP(+H) PNP (left bar at each concentration) or a non-silencing control PNP (right bar at each concentration) after 24, 28, 72 and 96 hours of incubation.

FIGS. 5A and 5B show the effect of the PNP containing HKP(+H) formulated with the modified Chk1-targeting siRNAs containing gemcitabine on MiaPaCa2 xenografts in mice (n=8). FIG. 5A shows mean (±SE) tumor volume in treatment and control groups. FIG. 5B shows the mean (±SD) body weight in treatment and control groups.

### DETAILED DESCRIPTION

Pharmaceutical compositions comprising modified Chk1-targeting siRNA molecules containing one or more gemcitabine moieties in place of cytidine residues are provided, together with methods of making and using the siRNA molecules to treat a variety of cancers, including triple negative breast cancer (TNBC). Synthetic, modified siRNA sequences targeting Chk1 are shown in Table 1.

TABLE 1
Modified, synthetic siRNA constructs

| Name | Sense Strand (SS) Sequence | Antisense Strand (AS) Sequence |
|---|---|---|
| Non- | 5′- | 5′- |
| Silencing | CGAGCAGGGUAUCGACGA | UUUGUAAUCGUCGAUACCCUGCU |
| | UUACAAA-3′ | CG-3′ |
| | (SEQ ID No. 1) | (SEQ ID No. 2) |
| PGN6 | 5′- | 5′- |
| | AAGAAAgAgau[GEM]UGUA | AuUGAuACAGAUCuCuuucuu[GEM] |
| | U[GEM]AAU-3′ | [GEM]dTdT-3′ |
| | (SEQ ID No. 3) | (SEQ ID No. 4) |
| PGN7 | 5′- | 5′- |
| | AAGAAAgAgau[GEM]uGuAu | AuUGAuACAGAuCuCuUuCuU[GEM] |
| | [GEM]Aau-3′ | [GEM]dTdT-3′ |
| | (SEQ ID No. 5) | (SEQ ID No. 6) |
| PGN6 | 5′- | 5′- |
| (no Gem) | AAGAAAgAgaucUGUAUcAA | AuUGAuACAGAUCuCuuucuudTdT- |
| | U-3′ | 3′ |
| | (SEQ ID No. 7) | (SEQ ID No. 8) |
| PGN7 | 5′- | 5′- |
| (no Gem) | AAGAAAgAgaucuGuAucAAu- | AuUGAuACAGAuCuCuUuCuUdTdT- |
| | 3′ | 3′ |
| | (SEQ ID No. 9) | (SEQ ID No. 10) |
| PGN7S | 5′- | 5′-LS- |
| | A*A*GAAAgAgau[GEM]uGu | A*u*UGAuACAGAuCuCuUuC*u*U |
| | Au[GEM]A*A*u-3′ | [GEM][GEM]dTdT-3′ |
| | SEQ ID No. 11 | SEQ ID No. 12 |

Abbreviations from Table 1:
Capital letters not in bold: unmodified bases; bold uppercase letters: 2′OMe-modified bases; bold lowercase letters: 2′-fluoro-modified bases;
[GEM]: gemcitabine nucleosides; *: phosphorothioate; LS: phosphate. Note that chk1-21-wt as referenced in the figures or elsewhere in this disclosure is the same as PGN6 without
gemcitabine and the siRNA is not chemically modified.

### Rationale for Gemcitabine as a Therapeutic

Gemcitabine remains a cornerstone of PDAC treatment in all stages of the disease [6, 18]. Despite suboptimal clinical effects primarily caused by molecular mechanisms limiting its cellular uptake, activation and efficacy, and the development of chemoresistance within weeks of treatment initiation, gemcitabine is also used to treat other cancers [19]such as non-small cell lung cancer [20], bladder cancer [21], ovarian [22] and breast cancer [23]. Gemcitabine alone has a good toxicity profile, with myelosuppression being the most common side-effect [19]. However, it must be administered by infusion to minimize toxicity to organs at the high doses required.

Administration of a nanoparticle containing gemcitabine incorporated into an siRNA sequence is expected to reduce systemic exposure to gemcitabine and reduce the toxic side effects observed [8, 24]. Furthermore, the synergism between CHK11 siRNA and gemcitabine [1-3] allows the concentration required for efficacy to be reduced—so overall the exposure to gemcitabine can be much lower in patients.

Gemcitabine is an approved therapeutic in treatment of various cancers and shows activity against pancreatic cancer [6]. It is a cytidine analog, where two fluorine atoms have replaced the hydroxyl on the ribose [7]. It acts as a nucleoside metabolic inhibitor that causes DNA damage and blocks the progression of cells through the G1/S phase boundary, however, it has poor bioavailability and, when used in patients, requires infusion in large doses, resulting in significant toxicity [8]. Checkpoint kinase 1 (CHK1) is an integral part of DNA repair and also regulates G1/S transition [9,10]. Inhibition of CHK11 may enhance sensitization to DNA-damaging agents via reducing ribonucleotide reductase levels, shown to be important for resistance to gemcitabine activity [11]. Ribonucleotide reductase is composed of the homodimeric RRM1 and RRM2 subunits that catalyze the conversion of ribonucleotides to deoxyribonucleotides (dNTPs). These are used in the synthesis of DNA during replication and repair. Consequently, CHK11 inhibition results in exhaustion of dNTP and enhanced DNA damage [4]. A small molecule inhibitor of CHK1 decreased RRM1 and 2 and increased γH2AX, an established biomarker for DNA double-strand breaks (also increased by gemcitabine treatment) [4]. CHK1 may be involved in gemcitabine resistance in cancer therapy and CHK1 inhibition can increase the cytotoxicity of gemcitabine by interfering with DNA damage checkpoints independent of p53 status in pancreatic cell lines [14].

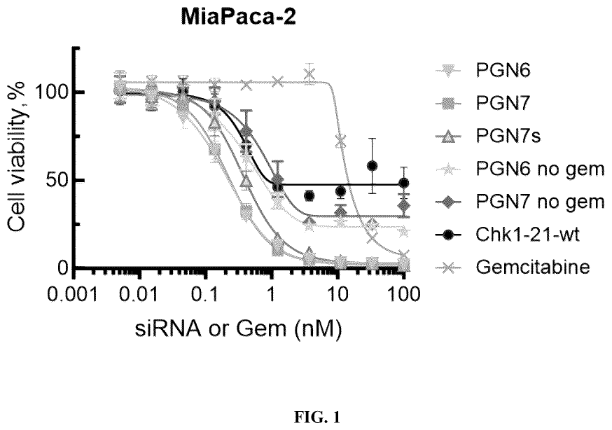

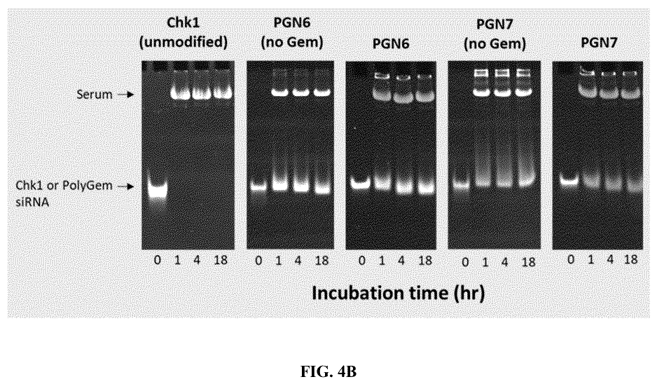

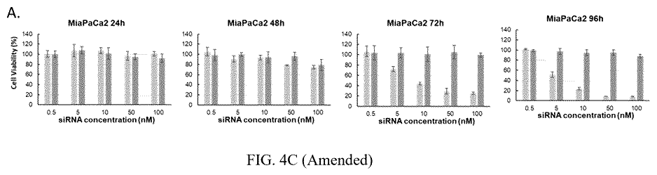

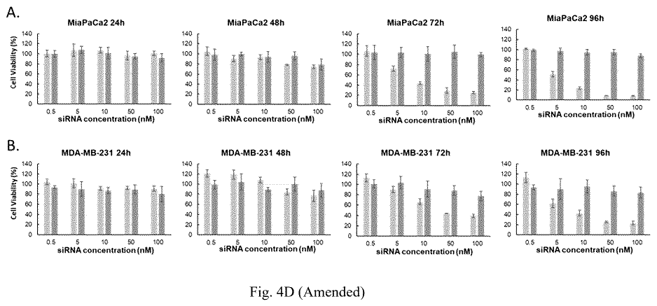

Notably, gemcitabine alone demonstrated much weaker activity than CHK1 siRNA gemcitabine constructs, giving an IC₅₀ above 10 nM in the in vitro studies using MiaPaca-2 cells (FIG. 1). Chemical modification of the siRNA improved the serum stability of the construct over the unmodified sequence and reduced the rate of cleavage of gemcitabine from the constructs compared to the unmodified siRNA (FIG. 1, FIGS. 4A-4B). Despite the variety of chemical modification patterns tested, no further improvement in potency emerged. PGN6 and PGN7 showed similar dose-response curves in all cells tested and therefore can be used interchangeably.

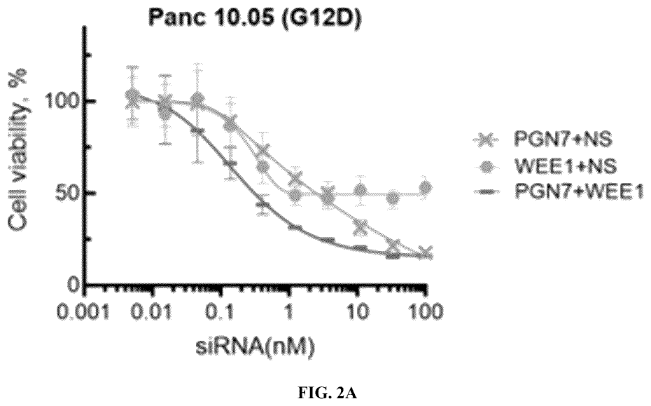

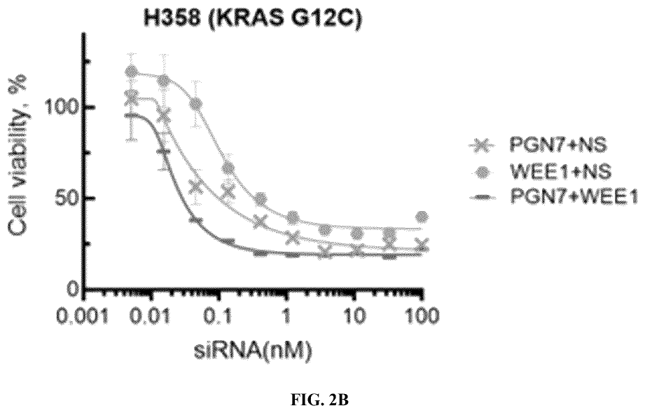

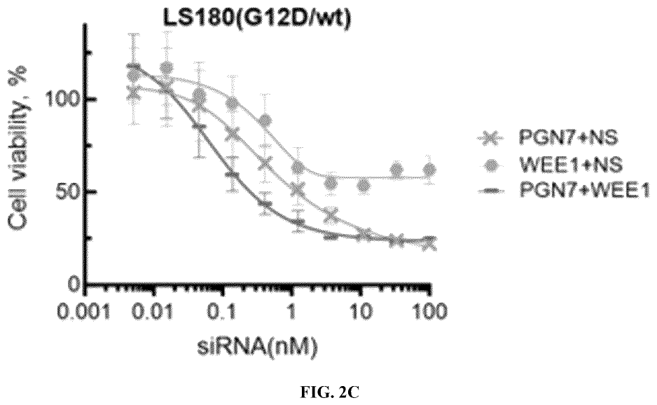

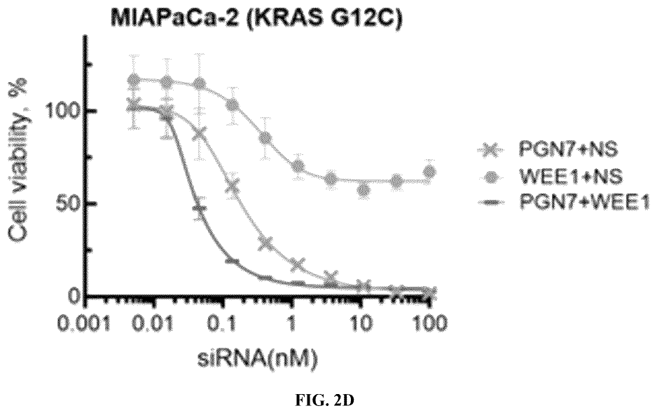

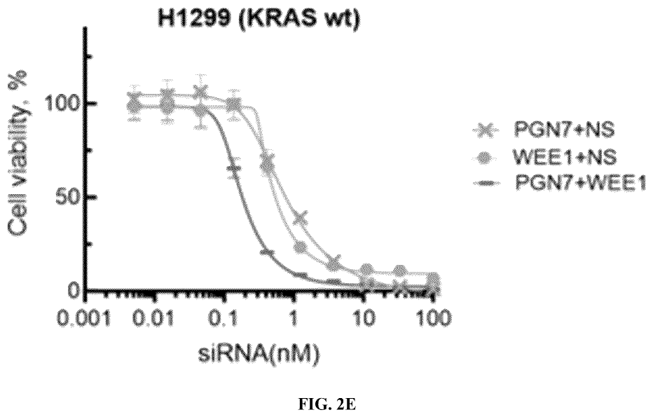

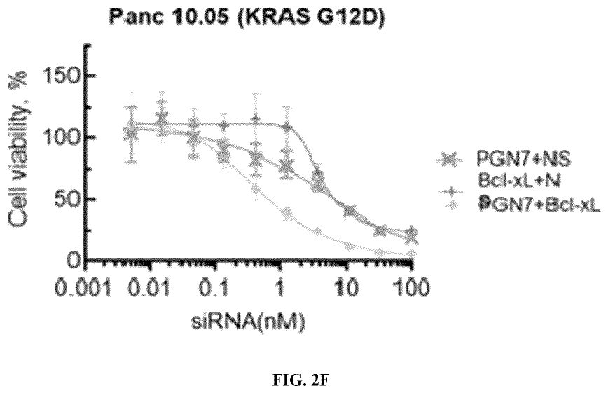

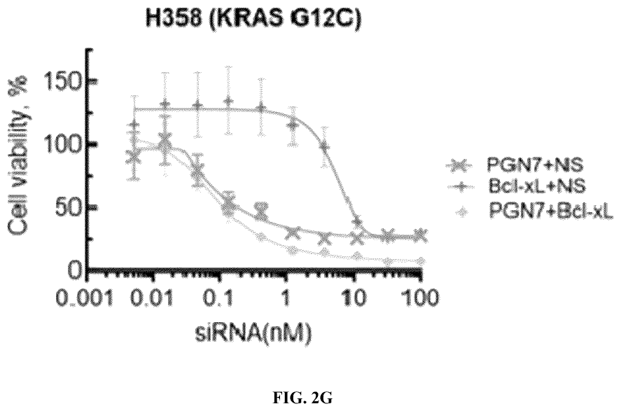

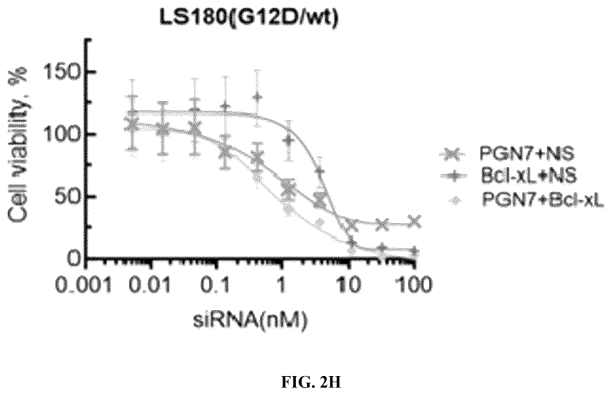

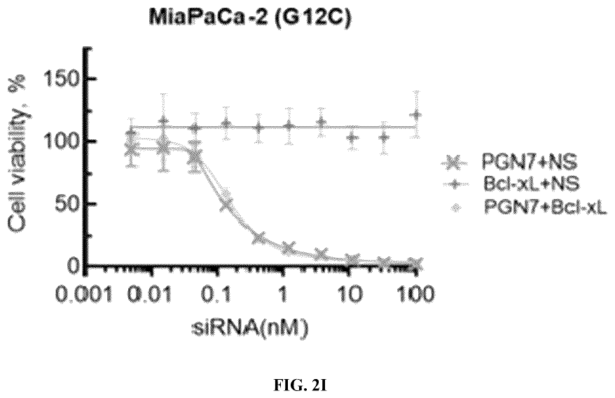

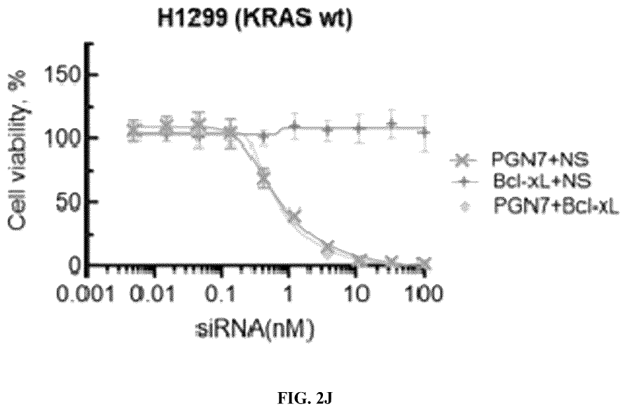

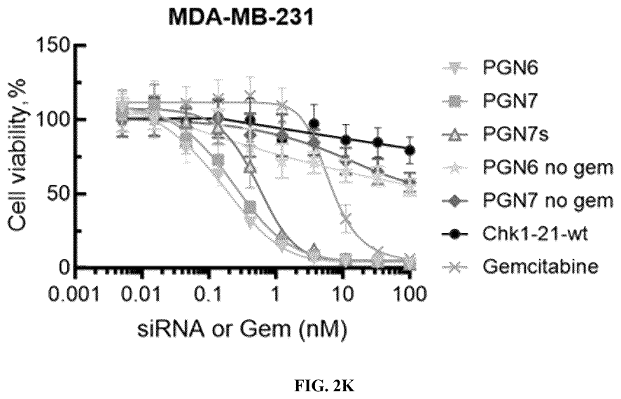

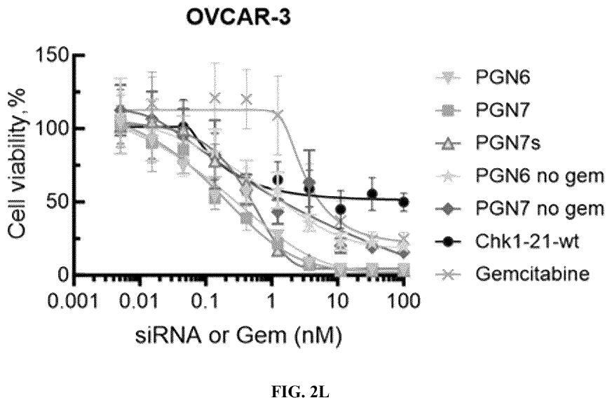

Previous findings [25] indicate that gemcitabine combinations could serve as a promising regimen for cancers other than pancreatic cancer. The combination of gemcitabine incorporated into CHK1 siRNA was studied in a variety of cancer cell lines. This included NSCLC with different KRAS backgrounds. H358 cells (KRAS G12C mutant cells) showed an IC50 of PGN7 of ~1 nM (FIGS. 2A-2E) while other cell lines showed higher IC50 values (e.g. H2030 (G12C) showed an IC50 of 3 nM; A549 (G12S) and H1299 (WT) showed an IC50 of ~6 nM).

Rationale for Combining polyGEM with WEE1 siRNA

CHK1 siRNA containing gemcitabine moieties demonstrated additivity with an siRNA against a second target (WEE1) in treating MiaPaca2 and BxPC3 pancreatic cells [1]. WEE1 has been shown to augment activity of CHK1 inhibition [9, 27-29] and be a relevant therapeutic target for cancer in its own right [26]. WEE1 regulates the G2/M checkpoint by catalyzing an inhibitory tyrosine phosphorylation of CDK2/cyclin B kinase complex and hence terminates the cell cycle. Inhibiting WEE1 has been shown to lead to DNA damage due to unchecked replication and potentiates the effect of DNA-damaging therapeutics [26]. WEE1 is downstream of CHK1 and therefore WEE1 siRNA will also increase the activity of gemcitabine. Consequently, inhibition of WEE1 using siRNA is expected to have a further antitumor effect—especially in the presence of gemcitabine and inhibition of CHK1 and possibly without a concomitant increase in in side effects or toxicity in normal tissues [38]. It was found that that Chk1-gemcitabine (PGN7) enhances the activity of WEE1 siRNA in a number of cell lines (FIGS. 2A-2E). This included pancreatic cancer cells, lung cancer cells and colon cancer cells. The siRNA sense strand (5′ to 3′) sequence used for WEE1 previously disclosed is: CGCTCTGTCAGCCTTACTA (SEQ ID No. 13)

In all cell lines, PGN7 and WEE1 siRNA provided an increase in potency compared to either siRNA alone and showed synergy as determined by the Combination Index (<1 for all cell lines). H358 (CI=0.83), Panc10.05 (CI=0.7), H1299 (CI=0.69), MiaPaca2 (CI=0.39) and LS180 (CI=0.33).

Combining polyGEM with Bcl-xL siRNA

A previous study [30] demonstrated that in 10 cell lines derived from different cancers, high Bcl-2 baseline expression was observed in cell lines that were resistant to gemcitabine (GEM-R). Gossypol treatment resulted in the decrease of anti-apoptotic genes such as Bcl-2 and Bcl-xL and an upregulation of the pro-apoptotic gene, Noxa, and demonstrated synergism with Gemcitabine in these tumor cells. The siRNA sense strand (5′ to 3′) sequence used for BCLxL previously disclosed is:

(SEQ ID NO. 14)
GCGTGGAAAGCGTAGACAAGGAGAT

Inhibition of Bcl-xL by siRNA was selective in augmenting the activity of PGN7 in the various cell lines studied (FIGS. 2F-2J). The combination increased efficacy in Panc10.05 cells had limited effect in H358 and LS180 cells (CI~1) but had little effect in MiaPaca2 cells or H1299 as determined from the Combination Index that suggested synergy of the combination in Panc10.05 (CI=0.28) and additivity in H358 (CI=0.95) and in LS180 (CI=1.09) and showed no additivity in MiaPaca2 or H1299.

Chemically Modified Bases in siRNA

Incorporation of chemically modified bases into siRNA can improve half-life of the product in serum (as shown here) and can improve efficacy against the target while minimizing off target effects [5]. A nanoparticle formulation using a HKP polypeptide to protect the siRNA in vivo may be used to deliver multiple siRNAs per particle. HKP(+H) has shown the ability to protect the siRNA during IV administration, efficacy against tumor xenografts upon in vivo administration [31-34, 36], and delivery of multiple siRNAs to a tissue [36].

Oligonucleotide therapeutics, such as those based on small interfering RNA (siRNA), are promising agents against pancreatic cancer, because they can identify a specific mRNA sequence and interfere with gene expression as molecular-targeted agents [12]. Consequently, RNAi therapeutics have been formulated with nanocarriers to treat pancreatic tumors [13].

Azorsa [2] and Fredebohm [3] demonstrated that small molecule inhibitors and siRNA targeting CHK1 could synergize with the concomitant addition of free gemcitabine against pancreatic tumor cells in vitro. Incorporating gemcitabine nucleotides into the sequence of a chemically unmodified siRNA targeting CHK1 augmented the antitumor activity observed in vitro against various pancreatic cell lines [1]. Maximal activity, against various pancreatic tumor cells in vitro, was observed when 4 gemcitabine molecules were incorporated into the siRNA: 2 within the sense strand (gemcitabine replacing cytidines) and 2 at the 3′ end of the antisense strand [1]. The combination retained efficacy of the siRNA (the ability to reduce expression of CHK1), whilst also showing the efficacy of gemcitabine, and the combination showed synergistic effects—possibly via promoting CDK-dependent DNA damage and ribonucleotide reductase downregulation [4] and destabilizing the DNA replication apparatus [9].

In this disclosure chemical modifications of the CHK1 siRNA backbone (specifically incorporating 2′-Fluoro and 2′ O-Methyl nucleotides) are used to enhance its stability. The same nucleotide sequence in the siRNA targeting CHK1 was used as previously described but the locations of 2′-Fluoro and 2′-O-Me modified bases in 2 constructs: PGN6 and PGN7 was varied. 2′-O-Me modification of the ribose provides a bulkier option than 2′-Fluoro modification and provides better resistance to degradation by nucleases [14]. Too many of the natural base 2′-O-Me modifications can diminish siRNA activity against its target, but the modifications can also suppress siRNA-driven innate immune activation, enhance specificity for the target (increasing activity), and reduce toxicity due to off-target mediated effects [15]. 2′-O-Me may play a role in enhancing stability and shows tighter binding to mRNA [15]. No difference in potency or efficacy was observed for the constructs based on variation in the number of 2′-O-Me modified bases between PGN6 and PGN7.

Bases 9-11 on the sense strand were modified with 2′-Fluoro (since this corresponds to the cleavage location of the antisense strand [14, 16]. The 14th base in the antisense strand was modified with a 2′-Fluoro modification since this position does not tolerate 2′-O-Me changes [17]. Changes in the number of 2′-Fluoro bases (with higher numbers in PGN6 than in PGN7) were compared. While the siRNA could be sensitive to the location of these changes, PGN6 and PGN7 show very similar results in a number of the cell lines tested, so, besides stabilizing the construct against serum nucleases (FIGS. 4A-4B), there does not seem to be an added benefit of the number of modifications of one kind or another.

The efficacy of modified siRNA efficacy alone (no gemcitabine included) was studied and it was found that adding gemcitabine moieties improved the potency of the siRNA. Chk1-21-WT was not chemically modified, and did not contain gemcitabine, and it was not as potent as PGN6 or PGN7 (each containing four gemcitabine moieties) in any of the cells tested (FIG. 1 and FIGS. 2K-2L). PGN6 and PGN7 lacking gemcitabine showed reduced efficacy compared to the same molecules with gemcitabine, and gave very similar results to CHK1-21-WT (FIG. 1 and FIGS. 2K-2L).

Incorporation of phosphorothioates can further stabilize siRNA and protect the molecules from nucleases [15]. However, PGN7s (that also had Phosphorothioates (designated as PS, or as an asterisk, *) at each end of the SS and AS strand) showed weaker activity than either PGN6 or PGN7. Without being bound by theory this reduction may be due to slower degradation of the molecule, (to release the gemcitabine moieties), or due to slower separation of the strands to release the antisense strand and silence CHK1. The PS modification also increases hydrophobicity of the siRNA, allowing interaction with plasma proteins [15]. A branched polypeptide (HKP(+H)) was used that spontaneously forms nanoparticles about 80 nM in diameter around the siRNA. This nanoparticle protects the siRNA from interactions with plasma proteins and nucleases in vivo and may obscure any benefit of PS incorporation into the siRNA (FIGS. 4A-4B).

Chemically Modified Chk1-Gem siRNA Molecules Show Activity Against a Tumor Xenograft In Vivo

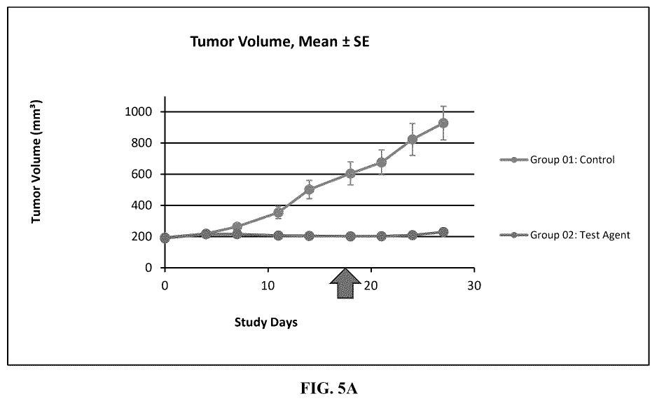

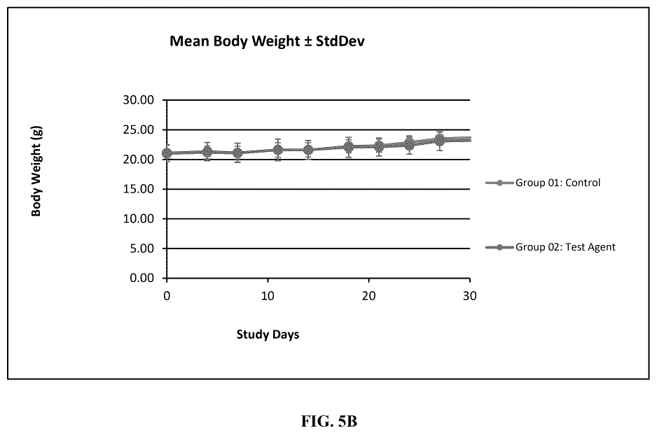

The constructs were tested for efficacy in vivo. A mouse xenograft model of MiaPaca2 cells was used, and PGN6 was formulated in a nanoparticle of HKP(+H). The product was injected IV through the tail vein of a mouse when the tumor reached a size of 200 mm³. The drug was injected at 2 mgs/kg BIW for 3 weeks and the results showed a significant inhibition in tumor growth (FIG. 5A) induced by the PGN6 formulation, while animals treated with a control (non-silencing) siRNA formulated in the same nanoparticle showed growth of the tumor over time. PGN6 nanoparticles showed no effect on body weights of the treated animals compared with the control treated animals (FIG. 5B).

### CONCLUSIONS

Robust activity of a chemically modified siRNA containing gemcitabine was demonstrated. The construct, targeting CHK1, showed improved activity (compared with gemcitabine alone) against a number of different tumor cell lines in vitro.

The chemically modified construct delivered to pancreatic xenografts, using a polypeptide nanoparticle that packages and protects multiple siRNAs per particle in vivo, demonstrated efficacy against the tumor. The inherent stability of the modified siRNA backbone, and resistance to degradation by serum, may allow direct conjugation of ligands allowing targeted delivery of the siRNA directly to tumor tissue.

### Definitions

As used herein, “a” or “an” may mean one or more. As used herein, “another” may mean at least a second or more.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52: 133-4), and derivatives thereof.

The term “copolymer” refers to a polymer that contains two or more types of units, regardless of the arrangement of units along the chain (random, alternating, block, graft), and regardless of its molecular structure (linear or branched).

The term “branch” is inclusive of any monomer or linear polymer (including co-polymer) thereof, which is covalently attached at least one end to the side group of a branching monomer. A branch which itself comprises one or more branching monomers is referred to as a “non-terminal branch”. A branch which does not comprise a branching monomer is referred to as a “terminal branch”. A “terminal branch” may include for example, the final division of branching of histidine or lysine to the n-terminal amino acid of the branch. The terminal branch may include a non-histidine or lysine amino acid (e.g., a cysteine or other linking agent), which aids in conjugating a stabilizing agent (such as PEG or HPMA) and/or a targeting ligand.

The term “branched polymer” is inclusive of any polymer comprising at least one backbone and at least one terminal branch. A branched polymer may further comprise one or more non-terminal branches.

The term “in vivo” includes therapy based on injection, whether intravenous or local (e.g., intratumoral, intramuscular, subcutaneous, intratracheal, intravenous, or intraocular injection into organ or airway directly, injection into vessels of the organ, or aerosolized into airways). The term “in vivo” also includes therapy based on electroporation of tumor, tissue, or organ.

The term “lipid” is used as it is in the art and includes any chemical species having a hydrophobic and a hydrophilic portion. Hydrophilic characteristics typically derive from the presence of phosphato, carboxylic, sulfato, amino, sulfhydryl, nitro, and other like groups. Hydrophobicity may be conferred by cholesterol and derivatives thereof and by the inclusion of groups that include, but are not limited to, long chain saturated and unsaturated aliphatic hydrocarbon groups and such groups substituted by one or more aromatic, cycloaliphatic or heterocyclic group(s).

The term “non-cationic lipid” refers to any of a number of lipid species that exist either in an uncharged form a neutral zwitterionic form, or an anionic form at physiological pH. Such lipids include, for example diacylphosphatidylcholine, diacylphosphatidylethanolamine, ceramide, sphingomyelin, cephalin, cardiolipin, cerebrosides, DOPE, and cholesterol.

The term “cationic lipid” refers to any of a number of lipid species which carries a net positive charge at physiologic pH. Such lipids include, but are not limited to, DODAC, DOTMA, DDAB, DOSPER, DOSPA, DOTAP, DC-Chol and DMRIE. Additionally, a number of commercial preparations of cationic lipids are available which can be used in the disclosed embodiments. These include, for example, LIPOFECTIN®. (commercially available cationic liposomes comprising DOTMA and DOPE, from GIBCO/BRL, Grand Island, N.Y., USA); LIPOFECTAMINE®. (commercially available cationic liposomes comprising DOSPA and DOPE, from GIBCO/BRL); and TRANSFECTAM®. (commercially available cationic liposomes comprising DOGS from Promega Corp., Madison, Wis., USA).

The term “Polydispersity Index,” or PDI, refers to the heterogeneity of a sample of nanoparticles. PDI is independent of nanoparticle size itself; rather the lower the PDI, the more homogeneous the size of nanoparticles in the sample. (ISO standards ISO 22,412, Particle size analysis—Dynamic light scattering (DLS)).

The term “peptide” is inclusive of both straight and branched amino acid chains, as well as cyclic amino acid chains, which comprise at least 2 amino acid residues. The terms “peptide” and “polypeptide” are used interchangeably herein.

A “pharmaceutical agent” includes any therapeutic agent useful in preventing, delaying or reducing the severity of the onset of a disease, or in reducing the severity of an ongoing disease, or in enhancing normal physiological functioning, as well as diagnostic agents, for example, a marker gene (GFP, luciferase). A “pharmaceutical agent” may consist of one or more therapeutic agents, one or more diagnostic agents, or a combination of one or more therapeutic and one or more diagnostic agents.

As used herein, a “pharmaceutically acceptable” component (such as a salt, carrier, excipient or diluent) of a pharmaceutical agent delivery composition according to the present disclosed embodiments is a component which (1) is compatible with the other ingredients of the delivery composition in that it can be included in the delivery composition without eliminating the capacity of the composition to deliver the pharmaceutical agent; and (2) where the delivery composition is intended for therapeutic uses, is suitable for use with an animal (e.g., a human) without undue adverse side effects, such as toxicity, irritation, and allergic response. Side effects are “undue” when their risk outweighs the benefit provided by the pharmaceutical agent.

As used herein, the term “physiologic pH” is defined as a pH between about 7.2 and about 7.5.

As used herein, the term “recombinant” means a cell having genetically engineered DNA, which was prepared in vitro and includes DNA from the host organism or, more often, from a different species, genus, family, order or class as compared to the host organism.

As used herein, an “siRNA molecule” is a duplex oligonucleotide, that is a short, double-stranded polynucleotide, that interferes with the expression of a gene in a cell that produces RNA, after the molecule is introduced into the cell. For example, it targets and binds to a complementary nucleotide sequence in a single stranded (“ss”) target RNA molecule, such as an mRNA, a micro RNA (miRNA) or short hairpin RNA (shRNA). The target RNA is then degraded by the cell. Such molecules are constructed by techniques known to those skilled in the art. Such techniques are described in U.S. Pat. Nos. 5,898,031, 6,107,094, 6,506,559, 7,056,704 and in European Pat. Nos. 1214945 and 1230375. By convention in the field, when an siRNA molecule is identified by a particular nucleotide sequence, the sequence refers to the sense strand of the duplex molecule. The term “siRNA” is used as it is in the art, and includes a duplex of RNA (19 to 25 bases or fewer in each strand) that targets mRNA. siRNA may be chemically or enzymatically synthesized. siRNA in accordance with the present disclosed embodiments may be incorporated and then activated in RISC (RNA-induced silencing complex).

A “therapeutically effective amount” is an amount necessary to prevent, delay or reduce the severity of the onset of disease, or an amount necessary to arrest or reduce the severity of an ongoing disease, and also includes an amount necessary to enhance normal physiological functioning. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to effect at least a 30% reduction in that parameter.

The word “transfect” is broadly used herein to refer to introduction of an exogenous compound, such as a polynucleotide sequence, into a prokaryotic or eukaryotic cell; the term includes, without limitation, introduction of an exogenous nucleic acid into a cell, which may result in a permanent or temporary alteration of genotype in an immortal or non-immortal cell line.

Determination of Efficacy of Modified Chk1 siRNAs with Gemcitabine

Depending on the particular targeted gene(s) and the selected nucleic acid (e.g., siRNA) sequence(s) and the dose administered, a partial or complete loss of targeted gene function may be observed. A reduction of mRNA levels or target protein expression (either mRNA expression or encoded polypeptide expression) in at least 50%, 60%, 70%, 80%, 90%, 95% or 99% or more of the targeted cells is exemplary. Degradation of mRNA levels or mRNA expression refers to the absence (or observable decrease) in targeted protein levels. Specificity refers to the ability to inhibit the translation of mRNA into proteins without manifesting effects on other genes in the targeted cells. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of target gene RNA sequence(s) by the nucleic acid molecules described in the disclosed embodiments also can be measured based upon the effect of administration of such molecules upon development/progression of an associated disease or disorder, e.g., tumor formation, growth, metastasis, etc., either in vivo or in vitro. Treatment and/or reductions in, e.g., tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., a 10-fold, 100-fold, 1000-fold, 105-fold, 106-fold, or 107-fold reduction in cancer cell levels could be achieved via administration of the pharmaceutical compositions containing them to cells, tissues, or to subjects in need. The subject may be a mammal, such as a human, a non-human primate or other animal used in a research, clinical or veterinary setting.

### Determination of Toxicity and Dosage

Toxicity and therapeutic efficacy of the pharmaceutical compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population) or IC₅₀ (the concentration of the composition which achieves a half-maximal inhibition of symptoms). The dose ratio between toxic and therapeutic effects is the therapeutic index and it can be expressed as the ratio LD₅₀/ED₅₀. Disclosed pharmaceutical compositions advantageously exhibit high therapeutic indices.

Data from cell culture assays and animal studies can be used in formulating a range of dosage for use in humans and other mammals. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ as determined in cell culture. Such information can be used to more accurately determine useful doses in humans. Levels in plasma may be measured, for example, by high performance liquid chromatography (HPLC). Advantageously, in some embodiments, the form of the pharmaceutical composition to be administered is a lyophilizate.

A therapeutically effective amount of a composition as described herein can be in the range of approximately 1 μg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the RNA contained in each sub-dose must be correspondingly smaller in order to achieve the total daily dosage unit. The dosage unit can also be compounded for a single dose over several days, e.g., using a conventional sustained release formulation which provides sustained and consistent release of the RNA over a several day period. Sustained release formulations are well known in the art. In this embodiment, the dosage unit contains a corresponding multiple of the daily dose. Regardless of the formulation, the pharmaceutical composition must contain RNA in a quantity sufficient to inhibit expression of the target gene in the animal or human being treated. The composition can be compounded in such a way that the sum of the multiple units of RNA together contain a sufficient dose.

The composition may be administered one or more times within a given period. The pharmaceutical compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

### Formulation of Pharmaceutical Compositions

The siRNA molecules containing gemcitabine moieties may be further formulated into pharmaceutical compositions using methods that are well known in the art. The composition may be formulated to be compatible with its intended route of administration, whether systemic or for local or regional effect, for example, parenteral, e.g., intramuscular, intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be advantageous to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. sterile powders for the preparation of sterile injectable solutions may be prepared using methods such as vacuum drying and freeze-drying, generating a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Methods of Administration and Treatment

Embodiments of the pharmaceutical compositions described herein may be used to treat a variety of disorders and diseases, including, e.g., various cancers, characterized by expression, and particularly altered expression, of genes targeted by the nucleic acid(s) (RNA), for example, triple negative breast cancer or pancreatic cancer.

Suitably formulated pharmaceutical compositions as described herein may be administered, as noted above, by means known in the art such as by parenteral routes, including intravenous, intramuscular, intraperitoneal, subcutaneous, intratumorally, transdermal, airway (aerosol), rectal, vaginal and topical (including buccal and sublingual) administration. Advantageously, the pharmaceutical compositions are administered by intravenous or intraparenteral infusion or injection. In the disclosed method embodiments, the compositions may be administered as described above and, advantageously may be delivered systemically or intratumorally. The compositions may be administered as a monotherapy, i.e., in the absence of another treatment, or may be administered as part of a combination regimen that includes one or more additional medications. For example, the compositions are used as part of a combination regimen for treating cancers includes an effective amount of at least one additional chemotherapy drug.

### EXAMPLES

The disclosed embodiments will be better understood by reference to the following examples which are intended for purposes of illustration and are not intended to be interpreted in any way to limit the scope of the appended claims.

The word “exemplary” is used herein to mean “serving as an example, instance, or illustration.” Any embodiment described herein as “exemplary” should not necessarily be construed as preferred or advantageous over other embodiments.

Similarly, it should be appreciated that in the above description of embodiments, various features are sometimes grouped together in a single embodiment, Figure, or description thereof to streamline the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 ¶6. It will be apparently to those having skill in the art that changes may be made to the details of the above-described embodiments without departing from the underlying principles of the disclosed embodiments.

While specific embodiments and application of the disclosed embodiments have been illustrated and described, it understood that the disclosed embodiments are not limited to the precise configuration and components disclosed herein. Various modifications, changes, and variations, which will be apparent to those skilled in the art may be made in the arrangement, operation, and details of the methods and systems of the embodiments disclosed herein, including those of the appended claims. Finally, various features of the disclosed embodiments herein may be combined to provide additional configurations, which fall within the scope of the disclosed embodiments. Some of the examples below illustrate the efficacy of certain of the disclosed embodiments of the inhibitory compounds.

### Example 1: Cell Viability Assay

All cell lines were obtained from ATCC (Rockville, MD). Cells were cultured in standard media supplemented with 10% FBS: Panc 10.05, H1299 and H358 in RPMI-1640 Medium, MiaPaCa-2, HT29 and MDA-MB-231 in DMEM, LS180 in EMEM. All media were obtained from ATCC (Rockville, MD).

Cells were seeded in 384-well plates at a density of 0.3×10³ to 1×10³ cells/well. On the next day, cells were treated with serially diluted gemcitabine or siRNAs delivered by Lipofectamine RNAiMAX (Life Technologies, Carlsbad, CA). Ninety-six (96) hours after addition of gemcitabine or transfection reagent, the number of viable cells was determined using CellTiter-Glo(R)-2.0 reagent (Promega, Madison, WI) by measuring luminescent signal on a Cytation 5 plate reader (BioTek Inc, Winooski, VT). All values were compared to values generated for cells treated with non-silencing (NS) siRNA or to non-treated control and reported as the percentage cell viability. Values represent the mean±SD (n=4). All values were normalized to values generated for cells treated with a non-silencing siRNA and reported as the percentage cell viability.

Calculation ofEC50 values. The EC50 values of the Gemcitabine, or Gemcitabine-modified siRNAs, (half-maximal effective concentration) were derived from a sigmoidal dose-response (variable slope) curve using GraphPad Prism 10.0.2(232) software (GraphPad Software, San Diego, Ca).

Combination index (CI) calculation. Synergism, additivity or antagonism in the different combinations was calculated on the basis of the multiple drug effect equation and quantitated by the combination index (CI) [37], where CI=1 indicates that the two drugs have additive effects, CI<1 indicates more than additive effects (“synergism”) and CI>1 indicates less than additive effects (“antagonism”). CI=(D)1/(Dx)1+(D)2/(Dx)2+(D)1(D)2/(Dx)1(Dx)2, where (Dx)1 and (Dx)2 are the doses of drug 1 and drug 2, alone, inhibiting 50%, whereas (D1) is the dose of drug 1 in combination, and (D2) the dose of drug 2 in combination that gives the experimentally observed 50% inhibition.

The siRNA sequence shown (Table 1) against CHK11 was prepared and several different chemical modification patterns of the siRNA backbone were evaluated, varying the locations of the 2′-Fluoro bases relative to the 2′-O-Me modifications and the incorporation of gemcitabine. Various constructs were examined where two gemcitabine moieties in the sense strand were augmented by inclusion of an additional two gemcitabine moieties at the 3′ end of the AS strand (See Table 1, PGN6 (SEQ ID Nos. 3-4), PGN7 (SEQ ID Nos. 5-6) and PGN7S (SEQ ID Nos. 11-12)). PGN7S also contained phosphorothioate groups at the ends of each strand.

These siRNA constructs were tested for their ability to reduce viability of pancreatic cancer using Miapaca2 cells after lipofectamine transfection of the siRNAs at varying concentrations. All of the constructs could inhibit the viability of these cells by 100% at concentrations below 100 nM. The contribution of the gemcitabine within the constructs was studied by making and evaluating constructs lacking the gemcitabine. The sequences lacking gemcitabine were much less effective in MiaPaca2 cells (and in all cells tested). The initial CHK11 construct lacking chemical modifications (CHkl-21-wt, the same as PGN6 without gemcitabine and without chemical modifications) showed similar IC₅₀ value to the modified siRNAs lacking gemcitabine, but also showed reduced efficacy at higher doses, producing only a partial inhibition of cell viability at the higher concentrations. The modified siRNA molecules lacking gemcitabine, (PGN6 no gem and PGN7 no gem), also did not show good efficacy at higher concentrations. The dose response curves for PGN6 and PGN7 in MiaPaca2 cells overlapped and showed a 100-fold improvement over gemcitabine alone. FIG. 1 shows the results of the cell viability study using various chemically modified siRNA molecules with (PGN6, PGN7, PGN7s) and without (PGN6, no gem; PGN7, no gem) gemcitabine for targeting Chk1 in MiaPaCa-2 (pancreatic cancer) cells.

polyGem (PGN6 and PGN7) exhibited greater potency in both TNBC (MDA-MB-231) and ovarian cancer cells (OVCAR-3). The chemically modified Chk1-21 sequences (lacking gemcitabine moieties in the construct) were noticeably less effective in MDA-MB-231 and OVCAR-3 cells than constructs containing gemcitabine.

FIGS. 2K and 2L show cell viability under the same conditions with the same set of siRNA molecules in MDA-MB-231 (breast cancer) cells and OVCAR-3 (ovarian cancer) cells.

The effect of combining the modified CHK1 siRNA containing gemcitabine (polyGem) with other siRNAs against targets that, when silenced, were expected to augment the activity of the polyGem siRNAs. When siRNA against WEE1 was combined with polyGem an improvement in potency was seen compared with either agent alone in Panc 10.05 cells, MiaPaca2 cells, H358 cells and H1299 cells as well as in LS180 (Colon cancer) cells (FIGS. 2A-2E).

When polyGem was combined with an siRNA against BCL-XL, improved potency and efficacy of the combination in Panc 10.05 cells was seen. The combination had no effect in MiaPaca2 cells, or on potency in H358 but showed an improvement in efficacy. A slight improvement in efficacy (without any impact on potency) was observed in LS180 cells for the combination, and a slight reduction in potency of the combination was observed in H1299 cells compared with polyGem alone. FIGS. 2A-2E show the results of the cell viability study using PGN7+NS, WEE1+NS, or the combination of PGN7 and WEE1 siRNAs with gemcitabine in a variety of cancer cell types (Panc 10.05; H358; LS180, MiaPaCa-2 and H1299). FIGS. 2F-2J show the results of the cell viability study using PGN7+NS, BCLxL+NS, or the combination of PGN7 and BCLxL in the same five cell lines.

### Example 2: Manufacture of siRNA Molecules

The siRNA molecules were synthesized with DMT ON using standard RNA synthesis protocols and commercially available reagents on a Mermade 12 oligo synthesizer (LGC). Dimethylformamidine (dmf) protected G-phosphoramidites were utilized for the synthesis of gemcitabine-containing siRNA molecules, facilitating milder cleavage and deprotection conditions. Cleavage of gemcitabine-containing RNA molecules was completed at room temperature using 1 mL 2N ammonium hydroxide in methanol for 2 hours. Subsequently, 1 mL 28-30% ammonium hydroxide solution was added and cleaved siRNA molecules were incubated at 45° C. for 16 hours to ensure complete deprotection of exo-amine groups. For RNA molecules without gemcitabine, cleavage and deprotection were achieved simultaneously using an AMA solution (1:1 28-30% ammonium hydroxide: methylamine) at room temperature for 2.5 hours. Purification of both gemcitabine- and non-gemcitabine-containing siRNA molecules was carried out following the Glen-pack DNA purification cartridge protocol, ensuring purity levels surpassing 85%. RNA purity was assessed using an anion exchange HPLC, and the identity of all single strands was confirmed via HRMS analysis. The RNA molecules containing phosphorothioate linkages tend to produce diastereomers of the same RNA, which could be seen as multiple peaks in HPLC analysis, but they result in a single most abundant mass peak. Quantification of siRNA molecules was performed using a UV-visible spectrophotometer. siRNA constructs were prepared in RNase-free water by heating sense and antisense strands in equimolar ratios at 90° C. for 5 minutes, followed by gradual cooling to room temperature over two hours. These meticulous syntheses, cleavage, deprotection, and purification techniques were employed to ensure the high quality and integrity of the siRNA constructs, making them suitable for biological investigations. Table 1 above shows the sense and antisense strands of the Chk1-targeting siRNA constructs containing gemcitabine, as well as the non-silencing siRNA constructs.

### Example 3: Formulation of Nanoparticles (PNP)

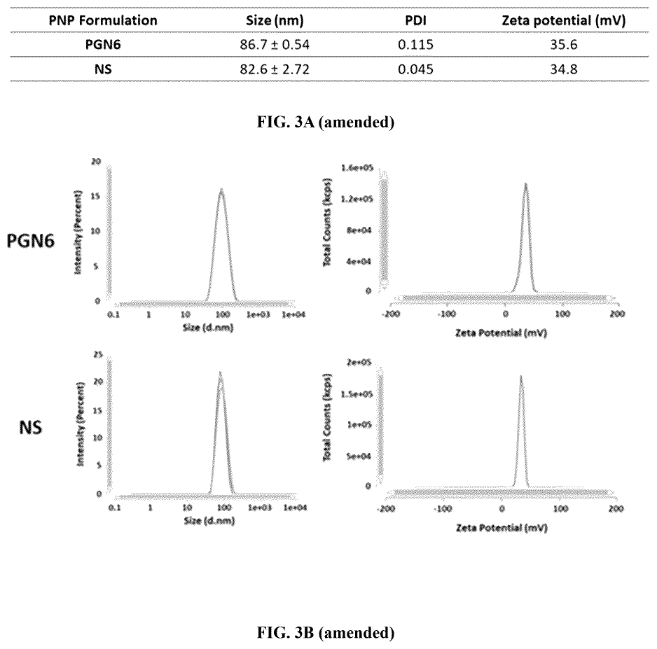

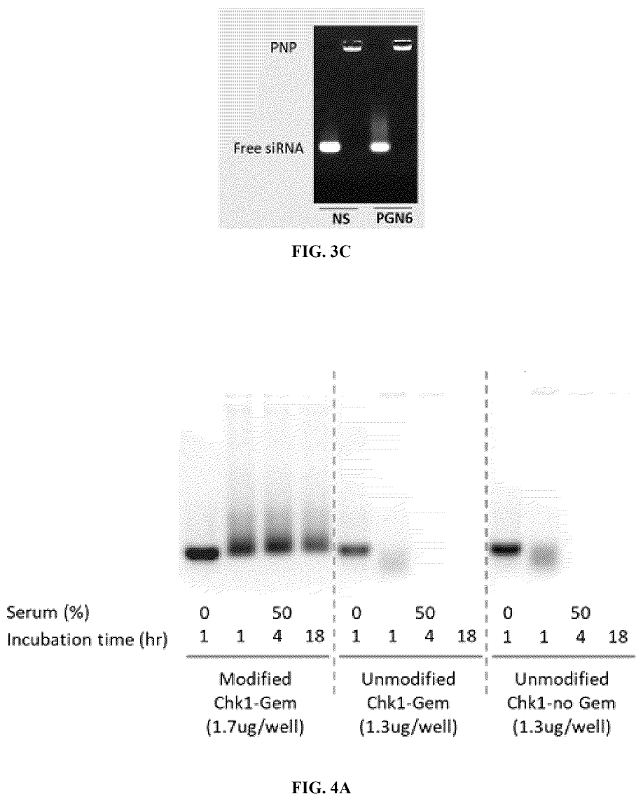

To test the efficacy of the products in vivo siRNAs (non-silencing siRNA (Control; NS), and PGN6) were formulated with a branched histidine lysine polymer (HKP (+H)) to form peptide nanoparticle (PNP) formulations were prepared using a microfluidic mixing system with a staggered herringbone patterned mixer (Precision NanoSystems, Vancouver, BC, Canada). HKP(+H) stock was prepared in water and diluted to appropriate concentrations. PolyGem siRNA or non-silencing (NS) siRNA stocks were prepared in water. The HKP(+H) and siRNA were mixed at a volume ratio of 1:1 at 8-10 mL/min total flow rate. Formulations were incubated for 30 min at room temperature. Particle size and zeta potential were then determined by dynamic light scattering with a Zetasizer Ultra (Malvern Panalytical, MA). A gel retardation assay was used to evaluate the efficiency of complex formation. Briefly, pre-formed PNPs were mixed with RNA loading dye and applied to 2% agarose gel. The gel was run in 0.5×TBE for 15 min. Electrophoretic mobility of the siRNA-nanoparticles was analyzed on a gel imaging system. The nanoparticles formed showed size characteristics between 82.6 nM (for NS) and 86.7 nM (for PGN6) and uniformity, as determined by low polydispersity index (PDI), using dynamic light scattering in a Malvern/Wyatt instrument (FIGS. 3A-3B). They also showed weakly positive charge (zeta potential of 34 mV for the non-silencing siRNA formulated in HKP(+H) and 35.8 mV for PGN6 in the same particles). FIG. 3C shows the results of the gel retardation assay.

### Example 4: Serum Stability Assay

The sensitivity of the siRNAs to nucleases present in serum was studied. The chemically modified siRNA were protected from degradation. The stability of the siRNAs was confirmed using polyacrylamide gel electrophoresis. The gemcitabine containing siRNAs (polyGem) were incubated with 90% (v/v) human serum for different time points at 37° C., and a sample was collected at 1, 4 and 18 hours. The siRNA without human serum was used as a control (0 h). After mixing the loading dye, mixtures of the siRNA and serum were loaded into 15% native polyacrylamide gel with 1.0×TBE. A PAGE gel was run for 60 min at 150 V. The PolyGem siRNAs in serum were analyzed on a gel imaging system (Azure 400, Azure biosystems). The siRNAs were stable for 18 h at 370° C. when treated with 50% serum. This contrasts with the unmodified siRNAs that showed rapid degradation by 50% serum (no siRNA was visible after a 4 h incubation). The 2′-Fluoro and 2′-O-Me modifications in the backbone of the siRNA protected the siRNA from serum while most of the unmodified siRNA was degraded in a little over an hour (FIG. 4A). PGN6 and PGN7 were compared with unmodified CHK1 siRNA after exposure to 90% serum. Unmodified CHK1 siRNA was completely degraded an hour after serum addition. Modified PGN6 or PGN7 was stable for 18 h in the presence of serum. The oligonucleotides were also studied without the gemcitabine moieties in the siRNA and this, too, showed insensitivity to 90% serum (FIG. 4B).

Time and dose-dependent killing of MiaPaca2 cells and MD-MB-231 cells in vitro was observed when the PGN6 was formulated with HKP(+H) to create nanoparticles (with the characteristics shown in FIG. 3A) and these were used to transfect the cells. Cell viability was determined by CellTiter-Glo cell viability assay at the conclusion of the incubation times (24, 48, 72 and 96 hours). The apparent potency was reduced because PNP may take longer to get siRNA into cells than lipofectamine, but even so was still ~5 nM IC₅₀ for PNP delivered PGN6 at 96 h in MiaPaca-2 cells in vitro (FIG. 4C) and a slightly greater IC₅₀ (>5 nM) for PNP delivered PGN6 in MDA-MB-231 cells in vitro (FIG. 4D).

### Example 5: In Vivo Assays

In vivo studies were performed at Crown Bio San Diego to examine the ability of nanoparticles to deliver the modified siRNAs to a xenograft tumor when the composition was administered intravenously (IV). Eight female NOD/SCID mice (Jackson Labs) were used per group. MiaPaCa-2 cells were cultured and, at time zero, 3×10⁶ cells were inoculated per mouse in 0.1 ml of 1:1 PBS:Matrigel. When tumors reached ~200 mm³, mice were randomized based on tumor volume and treated, by IV injection, with either control (non-silencing siRNA in PNP at 2 mgs/kg) or with the test agent (PGN6 siRNA in PNP at 2 mgs/kg). Tumor growth was monitored before every administration. Treatment was then provided for three weeks BIW throughout the experiment. A total of six doses were administered and body weight and tumor volume were each recorded twice per week. After 6 doses were administered BIW, treatment was halted at day 18 (see arrow in FIG. 5A) but measurements continued for the next week.

No regrowth of the tumor was observed at this time. PGN6 resulted in significant inhibition of tumor growth from the day of first administration. Treatment with PGN6 formulated in HKP(+H) had no effect on body weights of the treated animals suggesting no significant toxicity of the formulation (FIG. 5B).

### REFERENCES

[1] Simonenko et al., NAR Cancer, 2(3) (2020) doi: 10.1093/narcan/zcaa016.
[2] Azorsa et al., J Transl. Med., 7, 43-50 (2009).
[3] Fredebohm et al., J Cell Sci., 126, 3380-3389 (2013).
[4] Liang et al., Oncol. Reports, 39, 1322-1330 (2018).
[5] Behlke, Oligonucleotides, 18, 305-320 (2008).
[6] Burris et al., J Clin. Oncol. 15(6), 2403-2413 (1997).
[7] Ciccolini et al., Cancer Chemother Pharmacol. 78(1), 1-12 (2016).
[8] Russell et al., Mol Imaging Biol. 19(6), 885-892 (2017).
[9] Koh, et al., Cancer Res. 78, 3054-3066 (2018).
[10] Isono et al., Scientific Reports, 11, 10181 (2021).
[11] Minami et al., J Pharmacol. Sci. 127(3), 319-325 (2015).
[12] Takakura et al., Int. J. Mol. Sci. 20, 3331-3348 (2019).
[13] Kim et al., Pharmaceutics, 13, 1638 (2021).
[14] Chemikov et al., Int. J Mol. Sci. 24, 956 (2023).
[15] Hu et al., Sig Transduct Target Ther. 5, 101 (2020).
[16] Foster et al., Mol. Ther. 26, 708-717 (2018).
[17] Shmushkovich et al., Nucleic Acids Res. 46, 10905-10916 (2018).
[18] Amrutkar et al., Cancers, 9, 157-180 (2017).
[19] Toschi et al., Future Oncol. 1(1), 7-17 (2005).
[20] Crino et al., J. Clin. Oncol. 17(11), 3522-3530 (1999).
[21] Von der Maase et al., J Clin. Oncol. 18(17), 3068-3077 (2000).
[22] Fowler et al., Gynecol. Oncol. 90(2 Pt. 2), 21-23 (2003).
[23] Carmichael et al., J. Clin. Oncol. 13(11), 2731-2736 (1995).
[24] Zhang et al., Biomaterials. 34(13), 3447-3458 (2013).
[25] Xie et al., Oncotarget, 9, 7148-7161 (2018).
[26] Do et al., Cell Cycle, 12(19), 3159-3164 (2013).
[27] Guertin et al., Cancer Cell Int. 12, 45-57 (2012).
[28] Davies et al., Cancer Biol. Ther. 12, 788-796 (2011).
[29] Carrassa et al., Cell Cycle, 11, 2507-2517 (2012).
[30] Wong et al., PLoS ONE, 7(12), e50786. (2012).
[31] Leng et al., Cancer Gene Ther. 15(8), 485-495 (2008).
[32] Yan et al., Mol Cancer Ther. 7(6), 1355-1364 (2008).
[33] Chou et al., Cancer Gene Ther. 18(10), 707-716 (2011).
[34] Chou et al., Biomacromolecules. 14(3), 752-760 (2013).
[35] Zhou et al., Oncotarget. 8(46), 80651-80665 (2017).
[36] Kim et al., NAR Cancer. 6, 1-11 (2024) doi: 10.1093/narcan/zcad059.
[37] Zhao et al., Front. Biosci. (Elite Ed). 2, 241-249 (2010) doi: 10.2741/e86.
[38] Ashwell, S., DNA Repair in Cancer Therapy, Chapter 10, 211-234 (2012).

1. A pharmaceutical composition comprising a modified, interfering ribonucleic acid (RNAi) molecule that inhibits expression of Chk1, comprising gemcitabine moieties in the place of one or more cytidine residues, and further comprising a histidine-lysine copolymer carrier, wherein the RNAi is selected from the group consisting of siRNA, miRNA, and shRNA and mRNA.

2. The pharmaceutical composition according to claim 1, wherein the histidine-lysine copolymer carrier comprises HKP or HKP(+H).

3. The pharmaceutical composition according to claim 1, wherein the modified RNAi comprises a 2′-OMe modification, a 2′-fluoro modification and/or a phosphorothioate linkage.

4. The pharmaceutical composition according to claim 1, wherein the RNAi molecule comprises a pair of sense and antisense strands selected from the group consisting of: SEQ. ID Nos. 3 and 4; SEQ. ID Nos. 5 and 6; SEQ. ID Nos. 7 and 8; SEQ. ID Nos. 9 and 10; and SEQ. ID Nos. 11 and 12.

5. The pharmaceutical composition according to claim 4, wherein the pharmaceutical composition further comprises a second RNAi molecule comprising a sense strand of SEQ. ID Nos. 13 or SEQ ID NO:14.

6. The pharmaceutical composition according to claim 1, further comprising lipofectamine.

7. A pharmaceutical composition comprising a first siRNA that inhibits expression of Chk1 and a second siRNA that inhibits expression of WEE1,
wherein the first siRNA comprises sense and antisense strands selected from the group consisting: SEQ. ID Nos. 3 and 4; SEQ. ID Nos. 5 and 6; SEQ. ID Nos. 7 and 8; SEQ. ID Nos. 9 and 10; and SEQ. ID Nos. 11 and 12,
wherein said second siRNA comprises a sense strand comprising SEQ. ID Nos 13 or SEQ ID NO: 14,
wherein said siRNAs optionally comprise one or more modifications selected from the group consisting of 2′-OMe, 2′-F and phosphorothioate modifications, and
wherein said composition further comprises a histidine-lysine copolymer carrier.

8. A method of treating cancer in a subject, comprising:
administering to the subject a pharmaceutically effective amount of a pharmaceutical composition according to claim 1.

9. The method according to claim 8, wherein the cancer is selected from the group consisting of triple negative breast cancer, colon cancer, pancreatic cancer, lung cancer, ovarian cancer and urinary bladder cancer.

10. The method according to claim 8, wherein said pharmaceutical composition is administered by intravenous, intratumoral and/or subcutaneous injection.

11. The method according claim 8, wherein said pharmaceutical composition comprises lipofectamine.

12. The method according to claim 11, wherein the cancer is selected from the group consisting of triple negative breast cancer, colon cancer, pancreatic cancer, lung cancer, ovarian cancer and urinary bladder cancer.

13. A method according to claim 8, wherein said pharmaceutical composition comprises a pharmaceutically effective amount of an siRNA comprising SEQ. ID Nos. 5 and 6 and an siRNA have a sense strand comprising SEQ. ID No. 13, wherein the composition further comprises HKP(+H) and wherein the pharmaceutical composition is administered through intravenous or intratumoral injection.

14. A method of treating cancer in a subject comprising administering to the subject a pharmaceutically effective amount of a pharmaceutical composition comprising an siRNA comprising SEQ. ID Nos. 5 and 6 and an siRNA have a sense strand comprising SEQ. ID No. 14, wherein the composition further comprises HKP(+H), and wherein the pharmaceutical composition is administered through intravenous or intratumoral injection.

15. The method according to claim 8, wherein the subject is a mammal.

16. The method according to claim 16, wherein the subject is a human.

17. A method of treating cancer in a subject, comprising:
administering to the subject a pharmaceutically effective amount of a pharmaceutical composition according to claim 7.

18. The method according to claim 17, wherein the cancer is selected from the group consisting of triple negative breast cancer, colon cancer, pancreatic cancer, lung cancer, ovarian cancer and urinary bladder cancer.

19. The method according to claim 17, wherein said pharmaceutical composition is administered by intravenous, intratumoral and/or subcutaneous injection, and wherein said pharmaceutical composition optionally comprises lipofectamine.

20. The method according to claim 17, wherein the subject is a human.
